(19) United States
(12) Patent Application Publication
(10) Pub. No.: US 2020/0131504 A1
(43) Pub. Date: Apr. 30, 2020

(54) PLASMID LIBRARY COMPRISING TWO RANDOM MARKERS AND USE THEREOF IN HIGH THROUGHPUT SEQUENCING

(71) Applicant: TSINGHUA UNIVERSITY, Beijing (CN)

(72) Inventors: Xiao LIU, Beijing (CN); Zhichao XU, Beijing (CN); Xiaolin WEI, Beijing (CN); Zhongyi WU, Beijing (CN); Jue RUAN, Beijing (CN)

(21) Appl. No.: 15/128,557

(22) PCT Filed: Mar. 24, 2015
(86) PCT No.: PCT/CN2015/074981

§ 371 Date: Aug. 22, 2019

(30) Foreign Application Priority Data
Mar. 26, 2014 (CN) 201410116844.2

(51) Int. Cl. C12N 15/10; C40B 40/02; C40B 50/06

(52) U.S. Cl. CPC C12N 15/1065; C12N 15/1093; C40B 50/06; C40B 40/02

(57) ABSTRACT

Provided is a plasmid library comprising a DNA insertion site and two barcode sequences located upstream and downstream of the site. The combinations of two barcode sequences of any two plasmids selected from the library are different. Also provided is a method for high-throughput paired-end sequencing of an inserted DNA using the plasmid library.

### TECHNICAL FIELD

The present invention belongs to the field of genomics, and relates to a method for high-throughput paired-end sequencing of DNA fragments with plasmids barcoded with random sequences.

### BACKGROUND Whole Genome Shotgun Method based on the Next Generation

Sequencing (NGS) technologies rocketed the field of genomics in the last decade with the features of low cost and rapidness. Nevertheless, when the length of sequencing fragment is greater than 1 kb or even longer, current NGS technologies also reach the bottleneck of uncontrollability, error rate and cost. Due to the limitation of the length of the sequencing fragment, repeat sequences longer than 1 kb will not be effectively measured which produce gaps, thereby causing troubles in research areas of genome de novo assembly, haplotyping, metagenomics, etc.

Library construction of bacterial artificial chromosome (BAC) plasmids, yeast artificial chromosome (YAC) plasmids, Fosmids, Cosmids and the like not only provides long fragments of genomic DNA for paired-end sequencing with Sanger method, establishing inter-gap links and making up the shortcomings of lacking of reading in NGS, but also serves as a library to afford research materials at hand for genetics, biochemistry and molecular biology research of the species. The disadvantages of this technique are being extremely slow with Sanger sequencing and expensive.

### SUMMARY OF THE INVENTION

It is an object of the present invention to provide a plasmid library used for high-throughput paired-end sequencing of DNA fragments to be tested.

In the plasmid library provided in the invention, each plasmid is a double strand circular DNA molecule formed by ligating a plasmid backbone fragment and a DNA fragment having a specific structure, wherein said DNA fragment having a specific structure comprises barcode sequence 1, insertion site sequence of DNA to be tested and barcode sequence 2 sequentially from upstream to downstream;

for any two plasmids in said plasmid library, combinations of the barcode sequence 1 and the barcode sequence 2 are different from each other; and

in said plasmid library, said plasmid backbone fragment does not contain a sequence which is same as the insertion site sequence of DNA to be tested.

In one embodiment of the invention, both of the barcode sequence 1 and the barcode sequence 2 are random sequences. It is not required for the random sequence to have any biological function, for example, not transcripting to produce RNA, not expressing to produce protein, not binding to any RNA or protein as a cis-acting element.

In one embodiment of the invention, for any two plasmids in said plasmid library, the plasmid backbone fragment and the insertion site sequence of DNA to be tested are identical to each other.

Kinds of plasmids in said plasmid library are 100 or more.

Wherein, the combinations of the barcode sequence 1 and the barcode sequence 2 are different from each other can be understood as: for any two plasmids in the plasmid library, at least one of the two barcode sequences carried in one plasmid is different from that of the other plasmid, preferably both barcode sequences of one plasmid are different from that of the other plasmid.

Wherein, both lengths of the barcode sequence 1 and the barcode sequence 2 can be from 10 bp to 200 bp, for example, from 10 bp to 40 bp, and from 15 bp to 25 bp.

The insertion site sequence of DNA to be tested can be a recognition sequence of restriction site, an upstream or downstream homologous arm sequence used for homologous recombinant, other structural sequence for insertion of DNA to be tested, or a sequence formed by adding additional DNA sequences to each of the above sequence which can also be used for insertion of DNA to be tested. The length of the insertion site sequence of DNA to be tested can be from 4 bp to 1 Kb. When the insertion site sequence of DNA to be tested is a recognition sequence of restriction site, the length thereof is from 4 bp to 100 bp; when the insertion site sequence of DNA to be tested is an upstream or downstream homologous arm sequence used for homologous recombinant, the length thereof is from 50 bp to 1 Kb.

In one embodiment of the invention, particularly, the insertion site sequence of DNA to be tested is a recognition sequence of restriction site;

in each plasmid from said plasmid library, the sequence thereof apart from the recognition sequence of restriction site does not contain a restriction site corresponding to the recognition sequence of the restriction site.

The plasmid backbone fragment may be derived from a bacterial artificial chromosome plasmid, a yeast artificial chromosome plasmid, a Fosmid or a Cosmid.

In one embodiment of the invention, the plasmid backbone fragment is derived from a Fosmid named pcc2FOS plasmid. In particular, the plasmid backbone fragment is a fragment derived from a pcc2FOS plasmid by removing nucleotides 362 to 403 along with mutations A355C, T410G and A437G. Correspondingly, the added recognition sequence of restriction site is a sequence formed by ligating the recognition sequences of BamH I, Nhe I and Hind III sequentially.

In the plasmid library, the barcode sequence 1 and the barcode sequence 2 can all be composed of random sequences (the ordering of the nucleotides is random), or can be random sequences combined with specific sequences in various forms (e.g., contains a plurality of discrete random sequences of 1 bp or more). A principle in either case is that the theoretically possible combinations of said barcode sequence 1 and said barcode sequence 2 are more than 100. Dividing the plasmids of the plasmid library into more than 100 kinds (while said barcode sequence 1 and said barcode sequence 2 are different from each other in any two of the vast majority of plasmids) can meet the requirement of high-throughput sequencing.

It is another object of the present invention to provide a method for preparing said plasmid library.

The method for preparing the plasmid library provided by the invention may include the following steps (a) and (b), particularly:

(a) designing No.3 forward primer and No.3 reverse primer according to the following steps (al) to (a3):

(a1) designing No.1 reverse primer for amplifying a plasmid backbone fragment according to a sequence of upstream of site to be inserted or region to be substituted in original plasmid, and designing No.1 forward primer for amplifying a plasmid backbone fragment according to a sequence of downstream of the site to be inserted or the region to be substituted in the original plasmid;

(a2) ligating a sequence A with a length of 10-200 bp to the 5′-end of the No.1 reverse primer to obtain No.2 reverse primer; ligating a sequence B with a length of 10-200 bp to the 5′-end of the No.1 forward primer to obtain No.2 forward primer;

the sequence A and the sequence B are random sequences (the ordering of the nucleotides is random) or contain at least a plurality of discrete random sequences of 1 bp or more;

(a3) ligating a sequence C to the 5′-end of the No.2 reverse primer to obtain No.3 reverse primer; ligating a sequence D to the 5′-end of the No.2 forward primer to obtain No.3 forward primer;

the sequence C and the sequence D satisfy the following conditions: the 5′-end of the sequence C and the 5′-end of the sequence D each contains a restriction site K that is not present in the plasmid backbone fragment; and the 5′-end of the sequence C and the 5′-end of the sequence D are reverse complementary to each other; and the sequence C is a reverse complementary sequence of one strand at the 5′-end of the insertion site sequence of DNA to be tested; and the sequence D is a sequence of said one strand at the 3′-end of the insertion site sequence of DNA to be tested;

(b) using the original plasmid as a template for PCR amplification with the No.3 forward primer and the No.3 reverse primer, and the resulted PCR products were digested with endonuclease K and then self-ligated to obtain the plasmid library.

Wherein, after self-ligation of said PCR product, the method further comprises a step of transforming a recipient bacterium (e.g., Escherichia coli, particularly E. coli EPI300) with the ligation product, and then extracting plasmids from the transformed strain to obtain the plasmid library.

In step (a2) of said method, the lengths of said sequence A and said sequence B can further be 10-40 bp. In one embodiment of the invention, particularly, each of the lengths of the said sequence A and said sequence is 15-25 bp.

In step (a3) of said method, the insertion site sequence of DNA to be tested can be a recognition sequence of restriction site, an upstream or downstream homologous arm sequence used for homologous recombinant, or other structural sequence for insertion of DNA to be tested. The length of the insertion site sequence of DNA to be tested can be from 4 bp to 1 Kb. When the insertion site sequence of DNA to be tested is a recognition sequence of restriction site, the length thereof is from 4 bp to 100 bp; when the insertion site sequence of DNA to be tested is an upstream or downstream homologous arm sequence used for homologous recombinant, the length thereof is from 50 bp to 1 Kb.

The plasmid backbone fragment does not contain a sequence which is same as the insertion site sequence of DNA to be tested.

In one embodiment of the invention, particularly, the insertion site sequence of DNA to be tested is a recognition sequence of restriction site.

In the above method, the original plasmid is a bacterial artificial chromosome plasmid, a yeast artificial chromosome plasmid, a Fosmid or a Cosmid. In one embodiment of the invention, particularly, the original plasmid is a Fosmid named pcc2FOS plasmid. Correspondingly, the region to be substituted of the original plasmid is a sequence consists of nucleotides 362 to 403 of the pcc2FOS plasmid; the plasmid backbone fragment is a fragment derived from a pcc2FOS plasmid by removing nucleotides 362 to 403 along with mutations A355C, T410G and A437G; the recognition sequence of restriction site as the insertion site sequence of DNA to be tested is a sequence formed by ligating recognition sequences of BamH I, Nhe I and Hind III sequentially.

In one embodiment of the invention, particularly, step (a2) in the above method is:

ligating the following sequence to the 5′-end of the No.2 reverse primer to obtain No.3 reverse primer: a sequence formed by sequentially ligating recognition sequences of restriction sites Nhe I and BamH I (corresponding to the sequence C);

ligating the following sequence to the 5′-end of the No.2 forward primer to obtain No.3 forward primer: a sequence formed by sequentially ligating recognition sequences of restriction sites Nhe I and Hind III (corresponding to the sequence D).

In other words, the restriction site K is restriction site Nhe I.

Correspondingly, step (b) in the above method is: using the original plasmid as a template for PCR amplification with the No.3 forward primer and the No.3 reverse primer, and the resulted PCR products were digested with restriction enzyme (endonuclease) Nhe I and then self-ligated to obtain the plasmid library.

Use of said plasmid library in high-throughput sequencing of DNA fragments to be tested is also within the scope of the present invention.

In said use, the length of the DNA fragments to be tested can be from 15 kb to 400 kb.

In addition, linearized plasmid library satisfying the following conditions is also within the scope of the present invention:

sequences of linearized fragments obtained by linearization of the insertion site sequences of DNA to be tested in the plasmid library provided by the present invention are same as sequences in the linearized plasmid library.

It is yet another object of the present invention to provide a method for high-throughput sequencing of DNA fragments to be tested using said plasmid library or said linearized plasmid.

The method for high-throughput paired-end sequencing of DNA fragments to be tested by using the plasmid library provided by the present invention, a flow chart thereof is shown in FIG. 1, and particularly, the method includes the following steps:

(1) designing forward primer A and reverse primer A as follows:

designing forward primer 1 according to a sequence of the 3′-end of the plasmid backbone fragment; designing reverse primer 1 according to a sequence of the 5′-end of the plasmid backbone fragment; ligating an adaptor sequence 1 used for high-throughput sequencing to the 5′-end of the forward primer 1 to obtain forward primer A; and ligating an adaptor sequence 2 which is used in pair with the adapter sequence 1 to the 5′-end of the reverse primer 1 to obtain reverse primer A;

(2) using the plasmid library as a template for PCR amplification with the forward primer A and the reverse primer A to obtain PCR product 1; performing high-throughput sequencing of the obtained PCR product 1 according to the adapter sequence 1 and the adapter sequence 2 to obtain sequences of the barcode sequence 1 and the barcode sequence 2 of each plasmid in the plasmid library; pairing the barcode sequence 1 and the barcode sequence 2 existed in a same plasmid;

(3) cloning a batch of DNA fragments to be tested into the insertion site sequence of DNA to be tested of the plasmid library, wherein for each plasmid in the plasmid library, one of the DNA fragments to be tested is cloned into the plasmid; and transforming recipient bacterium with the obtained recombinant plasmid to obtain a DNA library;

(4) extracting the recombinant plasmid from the DNA library obtained in step (3) to obtain a recombinant plasmid library;

(5) performing following I) and II) in parallel:

I) digesting the recombinant plasmid library obtained in step (4) with restriction enzyme M; ultrasonic fragmenting; circularizing the fragmented DNA fragments to obtain circularized DNA molecular library 1;

II) digesting the recombinant plasmid library obtained in step (4) with restriction enzyme M′; ultrasonic fragmenting; circularizing the fragmented DNA fragments to obtain circularized DNA molecular library 2;

the restriction enzyme M and the restriction enzyme M′ satisfy the following conditions: the restriction enzyme M is located at the 3′-end of the plasmid backbone fragment in the plasmid library; the restriction enzyme M′ is located at the 5′-end of the plasmid backbone fragment in the plasmid library; and the distance from either enzyme to the barcode sequence 1 or the barcode sequence 2 is less than 10 kb;

the restriction enzyme M and the restriction enzyme M′ can be a same restriction enzyme or different restriction enzymes;

(6) designing forward primer B, reverse primer B, forward primer C and reverse primer C as follows:

designing forward primer 2 and reverse primer 2 according to the sequence of the 3′-end of the plasmid backbone fragment; designing forward primer 3 and reverse primer 3 according to the sequence of the 5′-end of the plasmid backbone fragment;

ligating an adaptor sequence 3 used for high-throughput sequencing to the 5′-end of the forward primer 2 to obtain forward primer B; ligating an adaptor sequence 4 which is used in pair with the adaptor sequence 3 to the 5′-end of the reverse primer 2 to obtain reverse primer B;

ligating the adaptor sequence 3 to the 5′-end of the forward primer 3 to obtain forward primer C; ligating the adaptor sequence 4 to the 5′-end of the reverse primer 3 to obtain reverse primer C;

(7) using the circularized DNA molecular library 1 obtained in step (5) as a template for PCR amplification with the forward primers B and the reverse primer B to obtain PCR product 2;

using the circularized DNA library 2 obtained in step (5) as a template for PCR amplification with the forward primers C and the reverse primer C to obtain PCR product 3;

performing high-throughput sequencing of the PCR product 2 and the PCR product 3 according to the adaptor sequence 3 and the adaptor sequence 4, respectively; obtaining the barcode sequence 1 and the 5′-end sequence of the DNA fragments to be tested in downstream thereof from the circularized DNA molecular library 1; obtaining the barcode sequence 2 and the 5′-end of DNA fragments to be tested in upstream thereof from the circularized DNA molecular library 2;

(8) determining sequences of both ends of each DNA fragment to be tested according to the pairing relationship between the barcode sequence 1 and the barcode sequence 2 obtained in step (2), thereby enabling high-throughput paired-end sequencing of the DNA fragments to be tested.

In step (3) of the method, the recipient bacterium can be Escherichia coli. In one embodiment of the present invention, the recipient bacterium is an E. coli DHI0b strain.

In the method, the high-throughput sequencing can be second-generation DNA sequencing. The adapter sequence used for high-throughput sequencing is determined based on the sequencer used. Specifically, the sequencers used in the present invention are Hiseq 2000 and Miseq manufactured by Illumina, Inc. Hiseq 2000 is used in high-throughput sequencing (first round of high-throughput sequencing) of step (1); Miseq is used in high-throughput sequencing (second round of high-throughput sequencing) of step (7). Correspondingly, adaptor sequences used are shown as follows: sequence of the adaptor sequence 1 and the adaptor sequence 3 is: 5′-AATGATACGGCGACCACCGAGATCTACACTCTTTCCCTACACG ACGCTCTTCCGATCT-3′ (SEQ ID NO: 1); sequence of the adaptor sequence 2 and the adaptor sequence 4 is: 5′-CAAGCAGAAGACGGCATACGAGATNNNNNNGTGACTGGAGTT CAGACGTGTGCTCTTCCGATCT-3′ (SEQ ID NO: 2) (wherein NNNNNN is the Illumina sequencing index which is a sequence used for distinguishing from other samples of upflow chamber in a same batch).

In step (5) of the method, particularly, “ultrasonic fragmentation” can be done with S220/E220 focused-ultrasonicator manufactured by Covaris, Inc. with a peak power of 105W and a duty cycle of 5% for 40 seconds. Particularly, “circularizing the fragmented DNA fragments” can be done by repairing both ends of the fragmented DNA fragment to blunt ends using an end repair enzyme (NEB), followed by ligating both ends of the DNA with T4 DNA ligase (NEB) to circularize.

In one embodiment of the invention, particularly, restriction enzyme M and restriction enzyme M′ in step (5) are both restriction enzyme Pvu II.

In the method, the length of the DNA fragments to be tested can be from 15 kb to 400 kb.

It is foreseeable to the person skilled in the art for the feasibility of the following method for high-throughput sequencing using the linearized plasmid library:

(I) ligating the DNA to be tested into the linearized plasmid library (e.g., Hind III) directly to construct the DNA library (corresponding to above step (3)); on one hand, performing high-throughput sequencing of the DNA library directly (corresponding to above steps (4)-(7)) to obtain the barcode sequence 1 and the 5′-end sequence of the DNA fragments to be tested in downstream thereof, and the barcode sequence 2 and the 3′-end sequence of the DNA fragment to be tested in upstream thereof; on the other hand, removing the DNA fragment to be tested which was ligated into the DNA library (e.g., using the same enzyme Hind III as in linearization), then circularizing the plasmid backbone to get an empty plasmid, and then performing high-throughput sequencing of the empty plasmid (corresponding to above steps (1)-(2)) to obtain the pairing relationship between the barcode sequence 1 and the barcode sequence 2;

(II) determining sequences of both ends of each of the DNA fragments to be tested according to the information obtained in the step (1), so as to achieve high-throughput paired-end sequencing of the DNA fragments to be tested.

The above method is also within the scope of the present invention.

It is prepared in the present invention a plasmid library barcoded with random sequences. Library constructed by such plasmid library not only has the characteristics of traditional library, but also can be used in high-throughput sequencing such as second-generation sequencing for the paired-end sequencing of genomic DNA therein. The present invention enables paired-end sequencing of long DNA fragments with the feature of rapidness, low-cost and accuracy.

### BRIEF DESCRIPTION OF THE DRAWINGS

FIG. 1 is a flow chart of high-throughput paired-end sequencing of DNA fragments to be tested provided by the present invention.

FIG. 2 is a schematic diagram showing a construction method of plasmid library barcoded with random sequences provided by the present invention.

FIG. 3 illustrates by taking BAC vector a of table 1 as an example, the sequences of both ends of the inserted fragment are matched to two sites on the chromosome IV of yeast genome, respectively; as is previously known from the sequencing of the empty vector, the random sequence barcodes ligated to the sequences of both ends of the inserted fragment are from the same vector, thus obtaining two paired sequences 153, 401 bp away from each other.

FIG. 4 is a plot of the results of high-throughput sequencing of 1536 yeast BAC libraries.

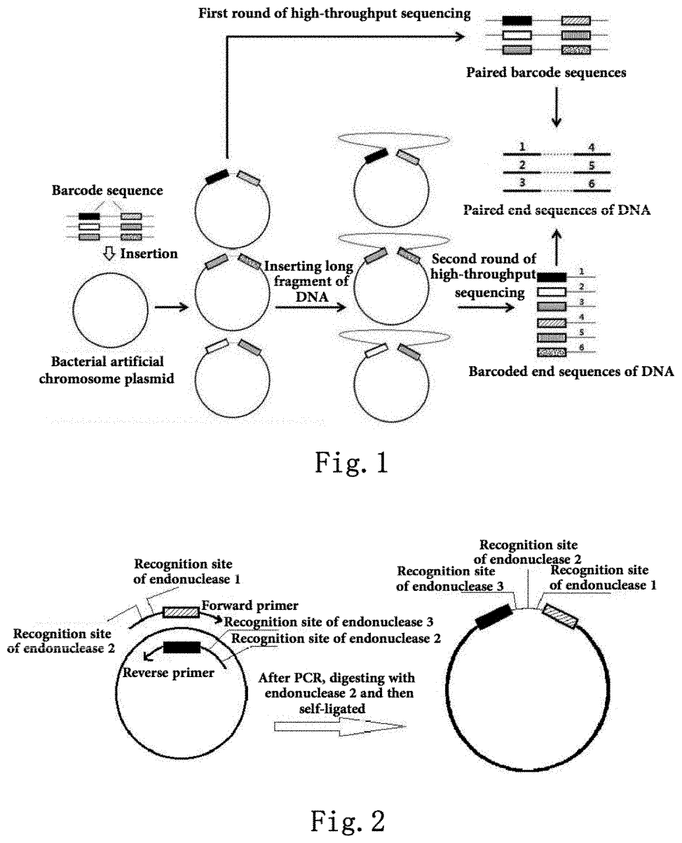

### DETAILED DESCRIPTION

The experimental methods used in the following examples are conventional methods unless otherwise specified.

The materials, reagents and the like used in the following examples are commercially available unless otherwise specified.

pcc2FOS Plasmid: product of Epicentre Corporation with catalog number ccfos059.

Yeast S288C: American Type Culture Collection (ATCC), No. 204508.

Escherichia coli EPI300: product of Epicentre Corporation with catalog number EC3001050.

Escherichia coli DH10b: product of Life Technologies Corporation with catalog number 18297-010.

### EXAMPLE 1.

### Preparation of Plasmid Library Barcoded with Random Sequences

In this embodiment, a pcc2FOS plasmid was used as an example to construct a plasmid library in which nucleotides 362 to 403 of the pcc2FOS plasmid was substituted by exogenous fragments containing random sequences. The details are as follows:

(1) Designing No.1 reverse primer for amplifying a plasmid backbone fragment according to a sequence of upstream of site to be inserted in pcc2FOS plasmid; and designing No.1 forward primer for amplifying a plasmid backbone fragment according to a sequence of downstream of the site to be inserted in pcc2FOS plasmid.

(2) Ligating random sequences with a length of 15-25 bp to the 5′-end of the No.1 reverse primer and the 5′-end of the No.1 forward primer as barcodes, respectively, to obtain No.2 reverse primer and No.2 forward primer, respectively;

sequentially ligating recognition sequences of restriction sites Nhe I and BamH I to the 5′ end of the No.2 reverse primer to obtain No.3 reverse primer (the sequence is shown below); and sequentially ligating recognition sequences of restriction sites Nhe I and Hind III to the 5′ end of the No.2 forward primer to obtain No.3 forward primer (the sequence is shown below).

No.3 Forward Primer:

5′-TAGC-GCTAGC-AAGCTT-CC-(N)₁₅₋₂₅-GTGGGAGCCTCTAGA GTCG-3′ (the underlined parts are the recognition sequences of restriction sites Nhel and Hind III, the sequence following (N)₁₅₋₂₅ is the sequence of No.1 forward primer, and the bold italicized base G is the mutated base at the 410th position of the pcc2FOS plasmid).

No.3 Reverse Primer:

5′-CGAT-GCTAGC-GGATCC-(N)₁₅₋₂₅-GTGGGAGCCCCGGGTA-3′ (the underlined parts are the recognition sequences of restriction sites Nhe I and BamH I, the sequence following (N)₁₅₋₂₅ is the sequence of No.1 reverse primer, and the bold italicized base G is the mutated base at the 355th position of the pcc2FOS plasmid).

Wherein, (N)₁₅₋₂₅ represents a random primer sequence while N can be any nucleotide among A, T, C and G; and the subscripted 15-25 represents a number of bases in the random primer.

(3) First, using pcc2FOS plasmid as a template for PCR amplification with the forward mutated primer and the reverse mutated primer shown below to obtain mutated pcc2FOS.

Forward Mutated Primer:

5′-ttcctaggctgtttcctggtgggaGcctctagagtcgacctgcaggcatgcGagctt-3′ (the first uppercase G is the base G mutated from the base T at the 410^{th} position and the second uppercase G is the base G mutated from the base A at the 437^{th} position.)

Reverse Mutated Primer:

5′-gtctaggtgtcgttgtacgtgggaGccccgggtaccgagctc-3′ (the uppercase G is the reverse complementary base of the base C which is mutated from the base A at the 355^{th} position.)

Next, using mutated pcc2FOS plasmid as template for PCR amplification with the No.3 forward primer and the No.3 reverse primer of step (2). PCR product was cut out of the gel and retrieved for digestion with Nhe I. Finally, digestion products were self-ligated to obtain the plasmid library barcoded with random sequences (FIG. 2). Then the plasmids were transformed into E. coli EPI300 and stored at -80° C.

### EXAMPLE 2

### High-Throughput Paired-End Sequencing of Long Fragments of DNA to be Tested with the Plasmid Library Prepared in Example 1

In this embodiment, the long fragments of DNA to be tested are from genome of yeast strain S288C (http://downloads.yeastgenome.org/sequence/S288C_reference/genome_releases/S288C_reference_genome_Current_Release.tgz).

1. First round of high-throughput sequencing

The sequencer is Illumina Hiseq 2000.

(1) Designing forward primer 1 according to a sequence of upstream of site to be inserted in pcc2FOS plasmid; designing reverse primer 1 according to a sequence of downstream of site to be inserted in pcc2FOS plasmid; ligating an adaptor sequence 1 used for high-throughput sequencing to the 5′-end of the forward primer 1 to obtain forward primer A (the sequence is shown below); ligating an adaptor sequence 2 which is used in pair with the adapter sequence 1 to the 5′-end of the reverse primer 1 to obtain reverse primer A (the sequence is shown below);

Forward Primer A:

5′-AATGATACGGCGACCACCGAGATCTACACTCTTTCCCTAC ACGACGCTCTTCCGATCT-acgactcactatagggcgaat-3′ (SEQ ID NO: 5) (the sequence in uppercase letters is the adaptor sequence 1; and the sequence in lowercase letters is the sequence of forward primer 1.)

Reverse Primer A:

5′-CAAGCAGAAGACGGCATACGAGATNNNNNNGTGACTGGA GTTCAGACGTGTGCTCTTCCGATCT-cgccaagctatttaggtgagac -3′ (SEQ ID NO: 6) (the sequence in uppercase letters is the adaptor sequence 2; and the sequence in lowercase letters is the sequence of reverse primer 1.)

wherein, ‘NNNNNN’ of reverse primer A is the Illumina sequencing index (N can be A, T, C or G) which is a sequence used for distinguishing from other samples of upflow chamber in a same batch.

(2) Culturing the Escherichia coli EPI300 transgenic strain frozen in Example 1 containing the plasmid library in LB liquid medium and then extracting the plasmids. Using the obtained plasmids as a template for PCR amplification with the forward primer A and the reverse primer A to obtain a PCR product (random sequence-recognition sequence of restriction site-random sequence); performing high-throughput sequencing of the obtained PCR product according to the adapter sequence 1 and the adapter sequence 2 to obtain specific sequence information of the two random sequences of each plasmid in the plasmid library; pairing the two random sequences existed in a same plasmid to obtain the pairing relationship between different random sequences.

2. Constructing a library by inserting the long fragments of DNA to be tested

(1) Acquisition of long fragments of yeast genomic DNA: liquid cultured yeast S288C was collected; after digestion of cell walls yeast protoplasts were evenly embedded in gel plug having a low melting point. Protease K was used to remove proteins. The yeast-containing gel plug was pre-digested with restriction enzyme Hind III, and the determined reaction condition was with an enzyme concentration of 20 U/ml for 10 minutes at 37° C. Finally, yeast genomic DNA fragments with a length from 120 kb to 300 kb were retrieved by pulsed-field gel electrophoresis.

(2) Digesting the plasmid library prepared in Example 1 with restriction enzyme Hind III, and performing end-blunting treatment by dephosphorylation or partial blunting to obtain blunt ends which is unable to self-ligate. Then the long fragments of genomic DNA extracted in step (1) was added for ligation. The plasmids inserted with the long fragments of genomic DNA were transformed into E. coli DH10b to obtain the genomic BAC library of yeast S288C.

3. Second round of high-throughput sequencing

The sequencer is Illumina Miseq.

(1) Incubating E. coli of the entire BAC library together. Extracting plasmids inserted with the genomic fragments (randomly selecting another 11 plasmids and denoted as a-k, performing Sanger sequencing of such plasmids for the validation of the accuracy of the method of the present invention). The plasmids were firstly digested with restriction enzyme Pvu II (a recognition sequence of Pvu II restriction site is located at both the upstream and the downstream of site to be inserted in pcc2FOS plasmid, i.e., at 218 bp and 651 bp), and subjected to focused ultrasonicator (Covaris 5220/E220)with a peak power of 105W and a duty cycle of 5% for 40 seconds. Then the fragmented DNA fragments were repaired with an end repair enzyme (NEB) to blunt ends and followed by ligation of both ends of the fragment with T4 DNA ligase (NEB). Thus the circularized DNA molecular library was obtained.

(2) Designing forward primer 2 and reverse primer 2 according to a sequence of upstream of site to be inserted in pcc2FOS plasmid; designing forward primer 3 and reverse primer 3 according to a sequence of downstream of site to be inserted in pcc2FOS plasmid; ligating adaptor sequence 3 used for high-throughput sequencing to the 5′-end of the forward primer 2 to obtain forward primer B (the sequence is shown below); ligating adaptor sequence 4 which is used in pair with the adaptor sequence 3 to the 5′-end of the reverse primer 2 to obtain reverse primer B (the sequence is shown below); ligating the adaptor sequence 3 to the 5′-end of the forward primer 3 to obtain forward primer C (the sequence is shown below); ligating the adaptor sequence 4 to the 5′-end of the reverse primer 3 to obtain reverse primer C (the sequence is shown below).

Forward Primer B:

5′-AATGATACGGCGACCACCGAGATCTACACTCTTTCCCTAC ACGACGCTCTTCCGATCT-acgactcactatagggcgaat-3′ (SEQ ID NO: 7) (the sequence in uppercase letters is the adaptor sequence 3; and the sequence in lowercase letters is the sequence of forward primer 2.)

Reverse Primer B:

5′-CAAGCAGAAGACGGCATACGAGATNNNNNNGTGACTGGA GTTCAGACGTGTGCTCTTCCGATCT-aatcgccttgcagcacatcc-3′ (SEQ ID NO: 8) (the sequence in uppercase letters is the adaptor sequence 4; and the sequence in lowercase letters is the sequence of reverse primer 2.)

Forward Primer C:

5′-AATGATACGGCGACCACCGAGATCTACACTCTTTCCCTAC ACGACGCTCTTCCGATCT-ttccagtcgggaaacctgtc-3′ (SEQ ID NO: 9) (the sequence in uppercase letters is the adaptor sequence 3; and the sequence in lowercase letters is the sequence of forward primer 3.)

Reverse Primer C:

5′-CAAGCAGAAGACGGCATACGAGATNNNNNNGTGACTGGA GTTCAGACGTGTGCTCTTCCGATCT-cgccaagctatttaggtgagac-3′ (SEQ ID NO: 10) (the sequence in uppercase letters is the adaptor sequence 4; and the sequence in lowercase letters is the sequence of reverse primer 3.)

Wherein, in reverse primer B and reverse primer C, ‘NNNNNN’ is the Illumina sequencing index (N can be A, T, C or G) which is a sequence used for distinguishing from other samples of upflow chamber in a same batch.

(3) Using the circularized DNA molecular library obtained in step (1) as a template for PCR amplification with the primer pair consisting of the forward primer B and the reverse primer B, and with the primer pair consisting of the forward primer C and the reverse primer C, respectively, to obtain PCR products; and performing high-throughput sequencing of the obtained PCR products according to the adaptor sequence 3 and the adaptor sequence 4, respectively, to obtain the relationship between the random sequence barcodes and the end sequences of the long fragments of genomic DNA.

Finally, obtaining the sequences of both ends of each long fragment of DNA to be tested according to the pairing relationship between random sequence barcodes obtained in Step 1 and the relationship between the random sequences and the end sequences of the long fragments of genomic DNA.

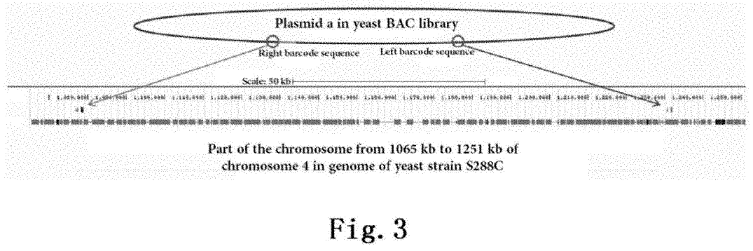

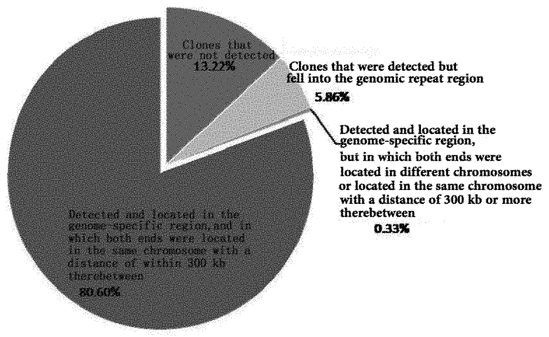

Taking the 11 BAC recombinant vectors denoted as a-k which were extracted from the genomic BAC library of yeast S288C obtained in Step 2 as examples, the sequencing results obtained by the second round of sequencing were compared with the yeast S288C genomic sequence through BLAST. The results showed that each random sequence in the 11 plasmids can correctly guide the pairing of the long fragments of genomic sequences ligated thereto. Except the insertion fragment of one BAC recombinant vector fell into the genomic repeat region, the insertion fragments of all other vectors were correctly mapped on to the genome of yeast S288C with normal fragment size. Detailed results are shown in Table 1 and FIG. 3.

TABLE 1
Comparison of sequencing results of the 11 BAC recombinant vectors

| | Random | | Position of | Position of | Length of |
|---|---|---|---|---|---|
| BAC | sequences | Chromo | left end of | right end of | insertion |
| Vector | on both ends | some | insertion | insertion | fragment |
| No. | paired or not | No. | fragment | fragment | (bp) |
| a | Yes | 4 | 1,231,584 | 1,078,183 | 153,401 |
| b | Yes | 14 | 147,194 | 277,470 | 130,276 |
| c | Yes | 4 | 1,399,204 | 1,231,996 | 167,208 |
| d | Yes | 7 | 669,525 | 837,576 | 168,051 |
| e | Yes | 3 | 243,852 | 108,723 | 135,129 |
| f | Yes | 7 | 200,433 | 34,847 | 165,586 |
| g | Yes | 8 | 203,862 | 332,736 | 128,874 |
| h | Yes | 7 | In repeat region around | | N/A |
| | | | 460,500 | | |
| i | Yes | 4 | 614,627 | 765,237 | 150,610 |
| j | Yes | 15 | 330,243 | 188,908 | 141,335 |
| k | Yes | 13 | 339,575 | 520,767 | 181,192 |

It can be seen that the plasmid library prepared in Example 1 of the present invention can perform high-throughput sequencing of the long fragments of DNA to be tested rapidly and accurately according to the method of Example 2.

### EXAMPLE 3

### Another Second Round of High-Throughput Sequencing of the Genomic BAC Library of Yeast S288C

The sequencer is Illumina Miseq.

(1) Incubating E. coli of the entire BAC library together. Extracting plasmids inserted with the genomic fragments. The plasmids were firstly digested with restriction enzyme Not I (a recognition sequence of Not I restriction site is located at both the upstream and the downstream of site to be inserted in pcc2FOS plasmid, i.e., at 3 bp and 686 bp), and subjected to focused ultrasonicator (Covaris S220/E220)with a peak power of 105W and a duty cycle of 5% for 40 seconds. Then the fragmented DNA fragments were repaired with an End Repair Enzyme (NEB) to blunt ends and followed by ligation of both ends of the fragment with T4 DNA ligase (NEB). Thus the circularized DNA molecular library was obtained.

(2) Designing forward primer 2 and reverse primer 2 according to a sequence of upstream of site to be inserted in pcc2FOS plasmid; designing forward primer 3 and reverse primer 3 according to a sequence of downstream of site to be inserted in pcc2FOS plasmid; ligating adaptor sequence 3 used for high-throughput sequencing to the 5′-end of the forward primer 2 to obtain reverse primer B (the sequence is shown below); ligating adaptor sequence 4 which is used in pair with the adaptor sequence 3 to the 5′-end of the reverse primer 2 to obtain reverse primer B (the sequence is shown below); ligating the adaptor sequence 3 to the 5′-end of the forward primer 3 to obtain forward primer C (the sequence is shown below); ligating the adaptor sequence 4 to the 5′-end of the reverse primer 3 to obtain reverse primer C (the sequence is shown below).

Forward Primer B:

5′-AATGATACGGCGACCACCGAGATCTACACTCTTTCCCTAC ACGACGCTCTTCCGATCT-acgactcactatagggcgaat-3′ (SEQ ID NO: 11) (the sequence in uppercase letters is the adaptor sequence 3; and the sequence in lowercase letters is the sequence of forward primer 2.)

Reverse Primer B:

5′-CAAGCAGAAGACGGCATACGAGATNNNNNNGTGACTGGA GTTCAGACGTGTGCTCTTCCGATCT-aagccagccccgacacc-3′ (SEQ ID NO: 12) (the sequence in uppercase letters is the adaptor sequence 4; and the sequence in lowercase letters is the sequence of reverse primer 2.)

Forward Primer C:

5′-AATGATACGGCGACCACCGAGATCTACACTCTTTCCCTAC ACGACGCTCTTCCGATCT-gcattaatgaatcggccaa-3′ (SEQ ID NO: 13) (the sequence in uppercase letters is the adaptor sequence 5; and the sequence in lowercase letters is the sequence of forward primer 3).

Reverse Primer C:

5′-CAAGCAGAAGACGGCATACGAGATNNNNNNGTGACTGGA GTTCAGACGTGTGCTCTTCCGATCT-cgccaagctatttaggtgagac-3′ (SEQ ID NO: 14) (the sequence in uppercase letters is the adaptor sequence 4; and the sequence in lowercase letters is the sequence of reverse primer 3.)

Wherein, in reverse primer B and reverse primer C, ‘NNNNNN’ is the Illumina sequencing index (N can be A, T, C or G) which is a sequence used for distinguishing from other samples of upflow chamber in a same batch.

(3) Using the circularized DNA molecular library obtained in step (1) as a template for PCR amplification with the primer pair consisting of the forward primer B and the reverse primer B, and with the primer pair consisting of the forward primer C and the reverse primer C, respectively, to obtain PCR products; and performing high-throughput sequencing of the obtained PCR products according to the adaptor sequence 3 and the adaptor sequence 4, respectively, to obtain the relationship between the random sequence barcodes and the end sequences of the long fragments of genomic DNA.

Finally, obtaining the sequences of both ends of each long fragment of DNA to be tested according to the pairing relationship between random sequence barcodes obtained in Step 1 and the relationship between the random sequences and the end sequences of the long fragments of genomic DNA.

High-throughput sequencing of 1536 yeast BAC libraries was performed according to the method described above. The results are shown below (see FIG. 4):

| | |
|---|---|
| Clones that were not detected | 203 |
| Clones that were detected but fell into the genomic repeat region | 90 |
| Detected and located in the genome-specific region, but in which | 5 |
| both ends were located in different chromosomes or located in | |
| the same chromosome with a distance of 300 kb or more | |
| therebetween | |
| Detected and located in the genome-specific region, and in which | 1238 |
| both ends were located in the same chromosome with a distance | |
| of within 300 kb therebetween | |
| In total | 1536 |

Sequences of both ends of 1251 BAC plasmids were obtained and compared with the genomic sequences. It was found that the barcode sequences of more than 99.8% plasmids can correctly guide the pairing of long fragment of genomic sequences ligated thereto.

1. A plasmid library, characterized in that:
each plasmid in the plasmid library is a double strand circular DNA molecule formed by ligating a plasmid backbone fragment and a DNA fragment having a specific structure, wherein said DNA fragment having a specific structure comprises barcode sequence 1, insertion site sequence of DNA to be tested and barcode sequence 2 sequentially from upstream to downstream;
for any two plasmids in said plasmid library, combinations of the barcode sequence 1 and the barcode sequence 2 are different from each other; and
in said plasmid library, said plasmid backbone fragment does not contain a sequence which is same as the insertion site sequence of DNA to be tested.

2. A method for preparing the plasmid library according to claim 1, comprising the following steps:
(a) designing No.3 forward primer and No.3 reverse primer according to the following steps (al) to (a3):
(a1) designing No.1 reverse primer for amplifying a plasmid backbone fragment according to a sequence of upstream of site to be inserted or region to be substituted in original plasmid, and designing No.1 forward primer for amplifying a plasmid backbone fragment according to a sequence of downstream of the site to be inserted or the region to be substituted in the original plasmid;
(a2) ligating a sequence A with a length of 10-200 bp to the 5′-end of the No.1 reverse primer to obtain No.2 reverse primer; ligating a sequence B with a length of 10-200 bp to the 5′-end of the No.1 forward primer to obtain No.2 forward primer; the sequence A and the sequence B are random sequences or contain a plurality of discrete random sequences of 1 bp or more;
(a3) ligating a sequence C to the 5′-end of the No.2 reverse primer to obtain No.3 reverse primer; ligating a sequence D to the 5′-end of the No.2 forward primer to obtain No.3 forward primer;
the sequence C and the sequence D satisfy the following conditions:
the 5′-end of the sequence C and the 5′-end of sequence D each contain a restriction site K that is not present in the plasmid backbone fragment; and
the 5′-end of the sequence C and the 5′-end of the sequence D are reverse complementary to each other; and the sequence C is a reverse complementary sequence of one strand at the 5′-end of the insertion site sequence of DNA to be tested; and the sequence D is a sequence of said one strand at the 3′-end of the insertion site sequence of DNA to be tested;
(b) using the original plasmid as a template for PCR amplification with the No.3 forward primer and the No.3 reverse primer, and the resulted PCR products were digested with endonuclease K and then self-ligated to obtain the plasmid library.

3. The plasmid library according to claim 1, characterized in that: both of the barcode sequence 1 and the barcode sequence 2 are random sequences.

4. The plasmid library according to claim 1, characterized in that: for any two plasmids in said plasmid library, the plasmid backbone fragment and the insertion site sequence of DNA to be tested are identical to each other.

5. The plasmid library according to claim 1, characterized in that: lengths of the barcode sequence 1 and the barcode sequence 2 are both from 10 bp to 200 bp.

6. The plasmid library or the method according to any one of claims 1-5, characterized in that: the insertion site sequence of DNA to be tested is a recognition sequence of restriction site;
the length of the recognition sequence of restriction site is from 4 bp to 100 bp.

7. The plasmid library or the method according to any one of claim 1-6, characterized in that:
the plasmid backbone fragment is derived from a bacterial artificial chromosome plasmid, a yeast artificial chromosome plasmid, a Fosmid or a Cosmid; or
the original plasmid is a bacterial artificial chromosome plasmid, a yeast artificial chromosome plasmid, a Fosmid or a Cosmid.

8. The plasmid library or the method according to claim 7, characterized in that:
the bacterial artificial chromosome plasmid is pcc2FOS plasmid; or
the plasmid backbone fragment is a fragment derived from a pcc2FOS plasmid by removing nucleotides 362 to 403 along with mutations A355C, T410G and A437G.

9. The plasmid library or the method according to claim 8, characterized in that:
the recognition sequence of restriction site is a sequence formed by ligating recognition sequences of BamH I, Nhe I and Hind III sequentially; or
in step (a3) of the method, the sequence C is a sequence formed by ligating recognition sequences of restriction sites Nhe I and BamH I sequentially; the sequence D is a sequence formed by ligating recognition sequences of restriction sites Nhe I and Hind III sequentially; or
in step (b) of the method, the endonuclease K is restriction enzyme Nhe I.

10. A linearized plasmid library, characterized in that: sequences in the linearized plasmid library are same as sequences of linearized fragments obtained by linearization of the insertion site sequences of DNA to be tested in the plasmid library according to any one of claim 1 and claims 3-9.

11. Use of the plasmid library or the linearized plasmid library according to any one of claim 1 and claims 3-10 in high-throughput paired-end sequencing of DNA fragments to be tested.

12. A method for high-throughput paired-end sequencing of DNA fragments to be tested by using the plasmid library or the linearized plasmid library according to any one of claim 1 and claims 3-10, comprising the following steps:
(1) designing forward primer A and reverse primer A as follows:
designing forward primer 1 according to a sequence of the 3′-end of the plasmid backbone fragment according to any one of claim 1 and claims 3-10; designing reverse primer 1 according to a sequence of the 5′-end of the plasmid backbone fragment; ligating an adaptor sequence 1 used for high-throughput sequencing to the 5′-end of the forward primer 1 to obtain forward primer A; ligating an adaptor sequence 2 which is used in pair with the adapter sequence 1 to the 5′-end of the reverse primer 1 to obtain reverse primer A;
(2) using the plasmid library according to any one of claim 1 and claims 3-10 as a template for PCR amplification with the forward primer A and the reverse primer A to obtain PCR product 1; performing high-throughput sequencing of the obtained PCR product 1 according to the adapter sequence 1 and the adapter sequence 2 to obtain sequences of the barcode sequence 1 and the barcode sequence 2 of each plasmid in the plasmid library; pairing the barcode sequence 1 and the barcode sequence 2 existed in a same plasmid;
(3) cloning a batch of DNA fragments to be tested into the recognition sequence of restriction site in the plasmid library, wherein for each plasmid in the plasmid library, one of the DNA fragments to be tested is cloned into the plasmid; and transforming recipient bacterium with the obtained recombinant plasmid to obtain a DNA library;
(4) extracting the recombinant plasmid from the DNA library obtained in step (3) to obtain a recombinant plasmid library;
(5) performing following I) and II) in parallel:
I) digesting the recombinant plasmid library obtained in step (4) with restriction enzyme M; ultrasonic fragmenting; circularizing the fragmented DNA fragments to obtain circularized DNA molecular library 1;
II) digesting the recombinant plasmid library obtained in step (4) with restriction enzyme M′; ultrasonical fragmenting; circularizing the fragmented DNA fragments to obtain circularized DNA molecular library 2;
the restriction enzyme M and the restriction enzyme M′ satisfy the following conditions: the restriction enzyme M is located at the 3′-end of the plasmid backbone fragment in the plasmid library; the restriction enzyme M′ is located at the 5′-end of the plasmid backbone fragment in the plasmid library; and the distance from either enzyme to the barcode sequence 1 or the barcode sequence 2 according to any one of claim 1 and claims 3-10 is less than 10 kb;
(6) designing forward primer B, reverse primer B, forward primer C and reverse primer C as follows:
designing forward primer 2 and reverse primer 2 according to the sequence of the 3′-end of the plasmid backbone fragment according to any one of claim 1 and claims 3-10; designing forward primer 3 and reverse primer 3 according to the sequence of the 5′-end of the plasmid backbone fragment;
ligating an adaptor sequence 3 used for high-throughput sequencing to the 5′-end of the forward primer 2 to obtain forward primer B; ligating an adaptor sequence 4 which is used in pair with the adaptor sequence 3 to the 5′-end of the reverse primer 2 to obtain reverse primer B;
ligating the adaptor sequence 3 to the 5′-end of the forward primer 3 to obtain forward primer C; ligating the adaptor sequence 4 to the 5′-end of the reverse primer 3 to obtain reverse primer C;
(7) using the circularized DNA library 1 obtained in step (5) as a template for PCR amplification with the forward primers B and the reverse primer B to obtain PCR product 2;
using the circularized DNA library 2 obtained in step (5) as a template for PCR amplification with the forward primers C and the reverse primer C to obtain PCR product 3;
performing high-throughput sequencing of the PCR product 2 and the PCR product 3 according to the adaptor sequence 3 and the adaptor sequence 4, respectively; obtaining the barcode sequence 1 and the 5′-end sequence of the DNA fragments to be tested in downstream thereof from the circularized DNA molecular library 1; obtaining the barcode sequence 2 and the 5′-end sequence of the DNA fragments to be tested in upstream thereof from the circularized DNA molecular library 2;
(8) determining sequences of both ends of each DNA fragment to be tested according to the pairing relationship between the barcode sequence 1 and the barcode sequence 2 obtained in step (2), thereby enabling high-throughput paired-end sequencing of the DNA fragments to be tested.


SEQUENCE LISTING (14 sequences)

SEQ ID NO: 1 - DNA, 58 bp, Artificial Sequence
  Adaptor sequence
aatgatacggcgaccaccgagatctacactctttccctacacgacgctcttccgatct

SEQ ID NO: 2 - DNA, 64 bp, Artificial Sequence
  Adaptor sequence
  misc_feature (25)..(30): “n” is a, c, g or t.
caagcagaagacggcatacgagatnnnnnngtgactggagttcagacgtgtgctcttccgatct

SEQ ID NO: 3 - DNA, 57 bp, Artificial Sequence
  Synthesized
ttcctaggctgtttcctggtgggagcctctagagtcgacctgcaggcatgcgagctt

SEQ ID NO: 4 - DNA, 42 bp, Artificial Sequence
  Synthesized
gtctaggtgtcgttgtacgtgggagccccgggtaccgagctc

SEQ ID NO: 5 - DNA, 79 bp, Artificial Sequence
  Forward primer
aatgatacggcgaccaccgagatctacactctttccctacacgacgctcttccgatctacgactcactatagggcgaat

SEQ ID NO: 6 - DNA, 86 bp, Artificial Sequence
  Reverse primer
  misc_feature (25)..(30): “n” is a, c, g or t.
caagcagaagacggcatacgagatnnnnnngtgactggagttcagacgtgtgctcttccgatctcgccaagctatttaggtgagac

SEQ ID NO: 7 - DNA, 79 bp, Artificial Sequence; identical to SEQ ID NO: 5
  Forward Primer

SEQ ID NO: 8 - DNA, 84 bp, Artificial Sequence
  Reverse Primer
  misc_feature (25)..(30): “n” is a, c, g or t.
caagcagaagacggcatacgagatnnnnnngtgactggagttcagacgtgtgctcttccgatctaatcgccttgcagcacatcc

SEQ ID NO: 9 - DNA, 78 bp, Artificial sequence
  Forward primer
aatgatacggcgaccaccgagatctacactctttccctacacgacgctcttccgatctttccagtcgggaaacctgtc

SEQ ID NO: 10 - DNA, 86 bp, Artificial Sequence; identical to SEQ ID NO: 6
  Reverse primer
  misc_feature (25)..(30): “n” is a, c, g or t.

SEQ ID NO: 11 - DNA, 79 bp, Artificial Sequence; identical to SEQ ID NO: 5
  Forward primer

SEQ ID NO: 12 - DNA, 81 bp, Artificial Sequence
  Reverse primer
  misc_feature (25)..(30): “n” is a, c, g or t.
caagcagaagacggcatacgagatnnnnnngtgactggagttcagacgtgtgctcttccgatctaagccagccccgacacc

SEQ ID NO: 13 - DNA, 77 bp, Artificial Sequence
  Forward primer
aatgatacggcgaccaccgagatctacactctttccctacacgacgctcttccgatctgcattaatgaatcggccaa

SEQ ID NO: 14 - DNA, 86 bp, Artificial Sequence; identical to SEQ ID NO: 6
  Reverse primer
  misc_feature (25)..(30): “n” is a, c, g or t.